(19) United States
(12) Patent Application Publication
(10) Pub. No.: US 2009/0088424 A1
(43) Pub. Date: Apr. 2, 2009

(54) METHODS AND COMPOSITIONS FOR CONTROLLING THE BIOAVAILABILITY OF POORLY SOLUBLE DRUGS

(76) Inventors: Ilan Zalit, Rosh Haain (IL); Anat Sofer-Raz, Herot (IL)

Correspondence Address: KENYON & KENYON LLP, ONE BROADWAY, NEW YORK, NY, 10004, US

(21) Appl. No.: 12/193,676

(22) Filed: Aug. 18, 2008

Related U.S. Application Data
(60) Provisional application No. 60/956,576, filed on Aug. 17, 2007.

(51) Int. Cl. A61K 31/538

(52) U.S. Cl. 514/230.5

(57) ABSTRACT

Provided are methods and compositions for controlling the bioavailability of poorly soluble drugs, including, for example, efravirenz.

### RELATED APPLICATIONS

This application claims the benefit under 35 U.S.C. 119(e) of U.S. Provisional Application No. 60/956,576, filed Aug. 17, 2007, which is incorporated herein by reference in its entirety.

### FIELD OF THE INVENTION

The invention encompasses methods and compositions for controlling the bioavailability of poorly soluble drugs, including, for example, efravirenz.

### BACKGROUND OF THE INVENTION

The absorption and bioavailability of any particular active pharmaceutical ingredient (“API”) can be affected by numerous factors when dosed orally. One particular example is an oral dosage form where the active drug has low water solubility. In such cases, the particle size, the solubility (of the API), and the formulation used play an important role in the dissolution and consequently the bioavailability of the API. Usually, good bioavailability of such drugs requires a formulation which enables a good dispersion of the API in the gastrointestinal fluids. In a dosage form containing a high dose of API (e.g., at least 300 mg), the dependence of bioavailability on formulation increases, mainly due to the limited amount of inactive ingredients (limited by the maximum size of an oral dosage form) which leads to a stronger tendency of the API to agglomerate within the manufacturing process of the dosage form.

The above mentioned factors may raise several difficulties in the production and the routine manufacturing of “poorly-soluble-high dose” dosage forms, as follows: The tendency of such drugs to agglomerate is strongly dependent on the chemical and physical properties of the active drug, e.g. polymorph, morphology, particle size, and OVI (organic volatile impurities) content. Further, the tendency of such drugs to agglomerate (as described above) may also result in high variability in the dissolution profile of the dosage form.

Therefore, one of the main challenges in the development of formulations with a high dose of poorly soluble drugs, is to develop a formulation that eliminates the tendency of the API to agglomerate, thus increasing the bioavailability of the drug. Accordingly, there is a need for formulations and methods of their preparation that effectively increase the bioavailability following administration of such drugs.

Efavirenz [(S)-6-chloro-4-(cyclopropylethynyl)-1,4-dihydro-4-(trifluoromethyl)-2H-3,1-benzoxazin-2-one] is a non-nucleoside HIV-1 reverse transcriptase inhibitor (NNRTI) approved by itself and in combination with other antiretrovirals for the treatment of HIV-1 infections. HIV-1 reverse transcriptase catalyzes the replication of viral RNA to render double stranded DNA and thus is a crucial element in the viral replication process.

Efavirenz is a crystalline nonhygroscopic lipophilic (log octanol water partition coefficient of 5.4) material with an aqueous solubility of 9.2 μg/ml (pH 8.7) at 25° C. There are several reported physical forms (I, II, III, IV, N, O, P, α, β, γ, γ1, γ2, φ, δ) as identified by X-ray diffraction. Forms I, II and III are polymorphs, and form IV is a heptane solvate. Various forms are described in U.S. Pat. No. 6,939,964 B2, WO 2006/040643 A2 and U.S. Pat. No. 6,673,372B1.

Efavirenz is reportedly disclosed in U.S. Pat. Nos. 5,519,021; 5,663,169; and 5,811,423. Efavirenz is sold by Bristol Myers Squibb under the tradename SUSTIVA® as a non-nucleoside HIV-1 reverse transcriptase inhibitor (NNRTI), and also under the tradename ATRIPLA® as a combination product including efavirenz, emtricitabine and tenofovir. It has been reported that peak efavirenz plasma concentration is reached 3 to 5 hrs after administration of the drug. The bioavailability of efavirenz is reportedly 40% to 45% and it may be administrated without regard to meals. However, administration with high fat meals should be avoided, as fat increases absorption. The elimination half-life for efavirenz is reportedly 40 to 55 hours after multiple doses, which support once-daily dosing.

WO 99/61026 describes preparation of a compressed tablet with comparable bioavailability to capsules using super-disintegrants and disintegrants intragranularly.

Makooi-Morehead et al. (U.S. Pat. Nos. 6,555,133 and 6,238,695) describe methods to enhance the dissolution rate of efavirenz from tablets or capsules by adding one or more super-disintegrants into the formulation. The addition of such super-disintegrating agent to the efavirenz formulation enhances the dissolution rate of efavirenz in the gastrointestinal tract thereby improving the rate and extent of absorption of efavirenz in the body.

WO 2006/134610 A1 describes preparation of a composition comprising efavirenz, sodium starch glycolate, PVP K-90, and lactose.

WO 2006/135933 describe the “triple” stable composition comprises efavirenz, emtricitabine, tenofovir DF and surfactant. The surfactant is said to have an important role in controlling the bioavailability of efavirenz.

### SUMMARY OF THE INVENTION

In one embodiment, the invention encompasses a method for producing a pharmaceutical composition of a poorly soluble active pharmaceutical ingredient, efavirenz, comprising comilling efavirenz with a hydrophilic polymer. Preferably the composition is then spray granulated. In one embodiment, the resulting composition does not contain a surfactant.

In one embodiment, the invention encompasses a pharmaceutical composition, comprising a co-milled composition of efavirenz with at least one hydrophilic polymer, wherein the efavirenz is present in an amount of at least 30% by weight of the pharmaceutical composition, excluding optional coating, wherein the pharmaceutical composition does not comprise a surfactant.

Preferably, at least about 15% of the total amount of efavirenz in the composition dissolves within about 15 minutes in a hydrophilic medium. More preferably, at least about 40% to about 50% of the total amount of efavirenz in the composition dissolves within about 30 minutes in the hydrophilic medium. Still more preferably, about 15% to about 40% of the total amount of efavirenz in the composition dissolves within about 15 minutes in the hydrophilic medium. Still more preferably, about 50% to about 70% or 55% to about 65% of the total amount of efavirenz in the composition dissolves within about 30 minutes in the hydrophilic medium. The above dissolution rates are determined using a solution of efavirenz in a hydrophilic medium having a concentration of about 3.17 ml of medium per each mg of efavirenz at 37° C.

In another embodiment, the pharmaceutical composition further comprises at least one carbonate or bicarbonate, and at least one pharmaceutically acceptable acid. The carbonate can be an alkali metal carbonate, e.g., sodium or potassium carbonate. The bicarbonate can be selected from the group consisting of ammonium bicarbonate, an alkali metal bicarbonate and an alkaline earth metal bicarbonate, such as sodium, magnesium, or potassium bicarbonate. The pharmaceutically acceptable acid can be selected from the group consisting of ascorbic acid, citric acid, tartaric acid, succinic acid, fumaric acid, malic acid, lactic acid, propionic acid, sorbic acid, and benzoic acid. In a preferred embodiment, the pharmaceutically acceptable acid is tartaric acid.

The invention also provides a method for treating or preventing a medical condition in a patient, comprising administering to said patient the pharmaceutical composition described above.

### BRIEF DESCRIPTION OF THE DRAWINGS

FIG. 1 illustrates in vitro dissolution rates of efavirenz compositions prepared by conventional wet granulation with two different efavirenz polymorphs compared to that of SUSTIVA®.

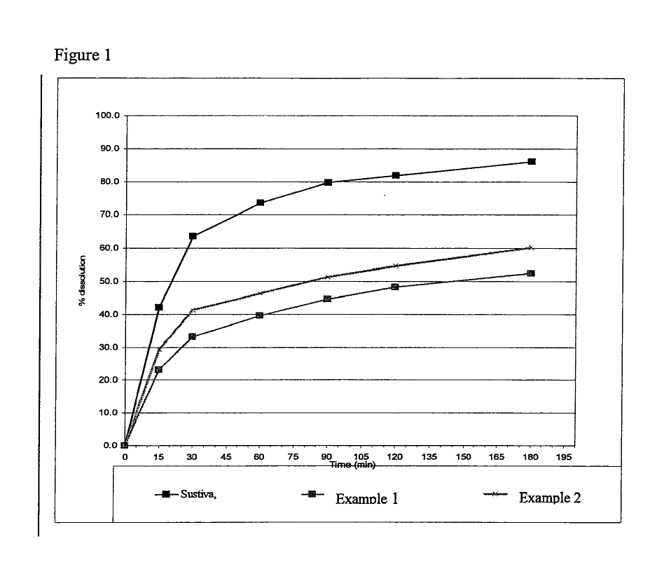

FIG. 2 illustrates in vitro dissolution rates of efavirenz compositions prepared by conventional dry granulation technique with two different efavirenz polymorphs compared to that of SUSTIVA®.

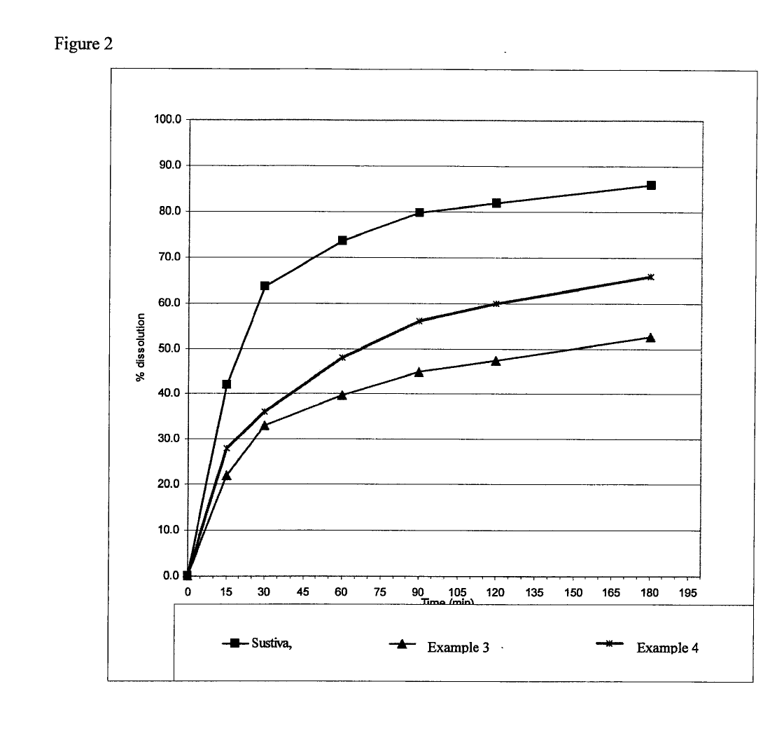

FIG. 3 illustrates in vitro dissolution rates of various efavirenz compositions prepared by wet milling and spray granulation method with various polymer amounts compared to that of SUSTIVA®.

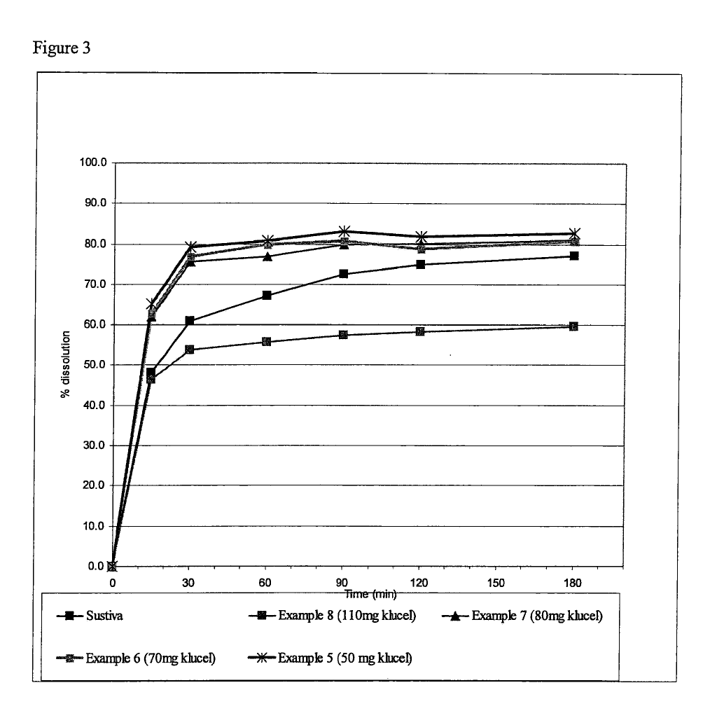

FIG. 4 illustrates in vitro dissolution rates of efavirenz compositions prepared by conventional wet granulation technique compared to that of ATRIPLA®.

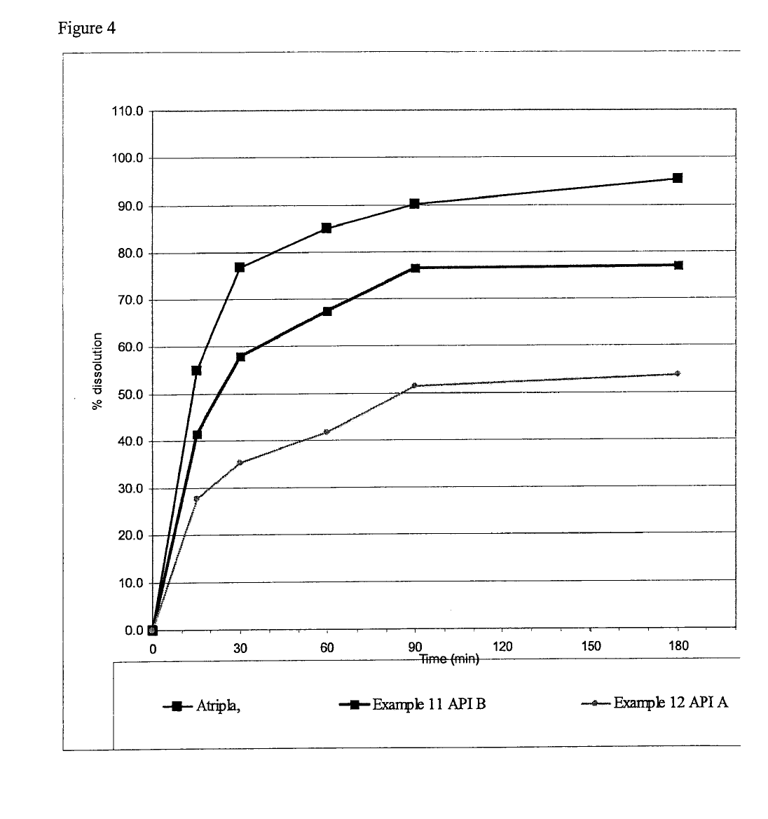

FIG. 5 illustrates in vitro dissolution rates of efavirenz in various compositions prepared by wet milling/spray granulation technique according to the invention, as compared to that of ATRIPLA®.

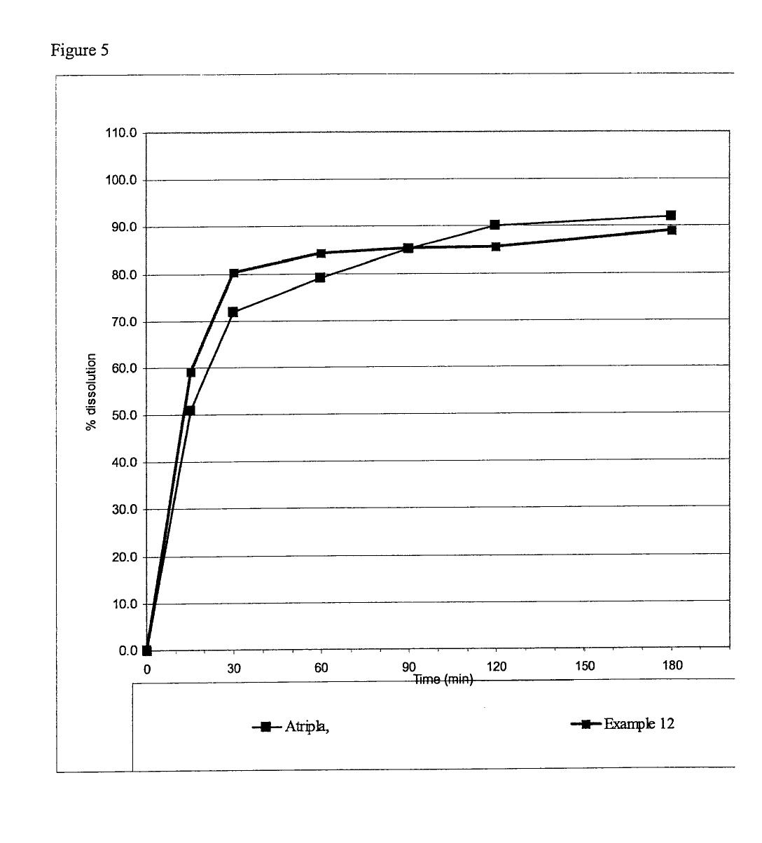

### DETAILED DESCRIPTION OF THE INVENTION

Some preferred embodiments of the invention provide methods and compositions for oral dosage forms containing a poorly soluble drug (efavirenz) in an amount of at least 30% by weight of the pharmaceutical composition, which preferably reduces the tendency of the efavirenz to agglomerate, and as a result increases the drug bioavailability, preferably without requiring a surfactant.

Other preferred embodiments of the invention provide methods and compositions that effectively control the bioavailability of a poorly soluble drug when administered at a high dose, e.g., in an oral pharmaceutical composition.

Other preferred embodiments provide a method for the production of such composition with a good manufacturing consistency of the product.

In a preferred embodiment, efavirenz is present in the pharmaceutical composition in an amount of at least about 30%, more preferably at least about 40%, by weight of the pharmaceutical composition, excluding coating (when present). For example, the efavirenz may be about 30-80%, about 30-70%, about 30-60%, about 30-50%, about 30-40%, about 40-80%, about 40-70%, about 40-60%, or about 40-50%. In another embodiment, the pharmaceutical composition comprises at least 300 mg of efavirenz, e.g., from about 300 mg to about 1000 mg, from about 300 to about 800 mg or from about 300 to about 600 mg. For example, the amount of efavirenz could be 300 mg, 400 mg, 500 mg, 600 mg, 700 mg, 800 mg, 900 mg or 1000 mg.

As used herein, unless otherwise defined, the term “poorly soluble,” when referring to an API, means that the API has a solubility of less than about 1.0 mg/ml.

As used herein, unless otherwise defined, the term “milling” includes reduction in size and/or de-agglomeration using techniques known in the art. For example, milling includes wet milling/homogenizing using homogenizer, such as rotor-stator and/or high pressure homogenizer such as a MICROFLUIDIZER®.

As used herein, unless otherwise defined, the term “co-milling” means the wet or dry milling of more than one compound. In a preferred embodiment of the invention, the process comprises co-milling efavirenz and at least one hydrophilic polymer and optionally one surfactant. For example, one compound may be a solid and a second compound may be dissolved or dispersed in a liquid, such as water. Co-milling in the absence of a surfactant is preferably done by wet co-milling. Wet co-milling is co-milling in the presence of a liquid, such as water or ethanol or other suitable liquids familiar to those skilled in the art.

In preferred embodiments co-milling and the use of a co-milled composition make it possible to improve the bioavailability of the API to a significantly greater extent compared with what is achieved either by milling of the API on its own, or by intimately mixing the separately micronized API and the excipient. Preferred pharmaceutically acceptable excipients are described below.

Suitable hydrophilic polymers include, but are not limited to, methacrylic acid co-polymer (e.g., EUDRAGIT® L, EUDRAGIT® E, EUDRAGIT® RS100 or EUDRAGIT® RL100), polyvinylpyrrolidone (“PVP”), polyethylene glycol (“PEG”), polyvinyl alcohol (“PVA”), vinylpyrrolidone/vinylacetate (“PVP-PVA”), hypromellose (hydroxypropylmethyl cellulose or “HPMC”) (e.g., PHARMACOAT®), hydroxypropylcellulose (“HPC”) (e.g., KLUCEL®), carboxymethylethylcellulose (“CMEC”), hydroxypropylmethylcellulose phthalate (“HPMCP”), hydrolyzed collagens (e.g., GELITA-COLLAGEL®). Preferably, the hydrophilic polymer is Klucel®. Preferably the hydrophilic polymer is present in an amount of about 0.2% to about 20%, about 0.5% to about 15%, about 0.5% to about 8%, about 0.5% to about 5%, or about 0.5% to about 2% by weight of the total composition. When the hydrophilic polymer is an HPC, such Klucel®, it is preferably present in an amount of about 0.5% to about 8%, more preferably in an amount of about 3% to about 4%. Any type of Klucel® (e.g., HF, MF, GF, JF, LF, EF) can be used in the present invention. However, preferably, the grade and amount of Klucel® used does not have a viscosity too high to adversely affect processing of the composition.

The method may optionally further comprise spray granulating wet milled API. The spray granulation may be performed by spraying a dispersion of the wet milled composition with a carrier in a fluid bed. The carrier may include any carrier known to one of ordinary skill in the art to be suitable for use in a pharmaceutical composition. In a fluid-bed, spray granulation process, particles and granulate are built up in a fluid bed by spraying a liquid onto fluidized particles. Thus in such process materials are fluidized in the fluid bed dryer and subsequently a solution is sprayed through a nozzle.

The pharmaceutical composition of the present invention may be prepared in any dosage form such as a compressed granulate in the form of a tablet for example. Also, uncompressed granulates and powder mixes that are obtained by the method of the present invention in the pre-compression steps can be simply provided in dosage form of a capsule or sachet. Therefore, dosage forms of pharmaceutical composition of the present invention include solid dosage forms like tablets, powders, capsules, sachets, troches and losenges. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant. Typically, the pharmaceutical acceptable excipient is that described above.

The pharmaceutical composition may further comprise at least one carbonate or bicarbonate, and/or at least one pharmaceutically acceptable acid. Suitable carbonates include, but are not limited to, alkali metal carbonates, such as sodium or potassium carbonates. Suitable bicarbonates include, but are not limited to, ammonium bicarbonate and alkali and alkaline earth metal bicarbonates, such as sodium, magnesium, or potassium bicarbonates. Suitable pharmaceutically acceptable acids include, but are not limited to, ascorbic acid, citric acid, tartaric acid, succinic acid, fumaric acid, malic acid, lactic acid, propionic acid, sorbic acid, and benzoic acid. Preferably, the pharmaceutically acceptable acid is tartaric acid.

Optionally, the pharmaceutical composition may further comprise at least one viscosity increasing agent. Suitable viscosity increasing agents include, but are not limited to, colloidal silicon dioxide, and alpha-lactose monohydrate (e.g., PHARMATOSE® and STARLAC™).

The pharmaceutical composition may optionally further comprise at least one additional pharmaceutically acceptable excipient. One of ordinary skill in the art will appreciate that any additional pharmaceutically acceptable excipient commonly used in the pharmaceutical industry may be used. The additional pharmaceutically acceptable excipient may include, for example, extragranular binders, disintegrants, glidants, lubricants, surfactants, preservatives, and antioxidants. In some embodiments, surfactants are excluded.

Extragranular binders typically bind the granulate and the other excipients after compression. Suitable extragranular binders include, but are not limited to, acacia, alginic acid, carbomer (e.g., CARBOPOL®), carboxymethylcellulose sodium, dextrin, ethylcellulose, gelatin, guar gum, hydroxyethyl cellulose, hydroxypropyl cellulose (e.g., KLUCEL®), hydroxypropyl methyl cellulose (e.g., METHOCEL®), liquid glucose, maltodextrin, methylcellulose, polymethacrylates, povidone (e.g., Povidone PVP K-30®, KOLLIDON®, and PLASDONE®), pregelatinized starch, sodium alginate, and starch.

Suitable disintegrants include, but are not limited to, croscarmellose sodium (e.g., AC-DI-SOL® and PRIMELLOSE®), crospovidone (e.g., KOLLIDON® and POLYPLASDONE®), microcrystalline cellulose, polacrilin potassium, powdered cellulose, pregelatinized starch, sodium starch glycolate (e.g., EXPOLTAB®, PRIMOJEL®), and starch.

Glidants typically improve the flowability of pre-compacted or un-compacted solid compositions and/or improve the accuracy of dosing during compaction and capsule filling. Suitable glidants include, but are not limited to colloidal silicon dioxide, magnesium trisilicate, powdered cellulose, and talc.

Lubricants typically improve the reduce adhesion and/or ease the release of the dosage form from, for example, dies and punches, during manufacturing. Suitable lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium stearyl fumarate, stearic acid, talc and zinc stearate.

Typical surfactants include, for example, sodium lauryl sulfate, poloxamers, polysorbate, PEG, and lecithin. In some preferred embodiments of the invention, no surfactant is present in the composition.

In one preferred embodiment, the invention encompasses a high-dose pharmaceutical composition comprising particles of efavirenz, wherein the surface of the particles are in contact with at least one dispersing agents. Preferably, the efavirenz particles are in contact with at least one of a binder and a polymer.

The pharmaceutical compositions described above may optionally include at least one additional API.

The pharmaceutical compositions described above may also be formulated into a solid dosage form. Suitable solid dosage forms include, but are not limited to, tablets and capsules. Preferably, the solid dosage form is a tablet.

Preferably, the dosage form exhibits rapid dissolution in a hydrophilic medium, which is a term well known in the art. Typically, it encompasses water and other aqueous solvents, such as the buffer described below. More preferably, the dosage form has a dissolution profile such that at least about 15% of the total amount of the API in the dosage form is dissolved within about 15 minutes and at least about 50% of the total amount of the API in the dosage form is dissolved within about 30 minutes under the following conditions: U.S.P. Type II (paddle) apparatus using 935 ml of buffer phosphate pH 6.0 with 0.15% sodium lauryl sulfate (SLS) at 37° C. at 50 rpm for a 300 mg dose of API. Or 1870 ml of buffer phosphate pH 6.0 with 0.15% SLS at 37° C. at 50 rpm for a 600 mg dose of API.

When the solid dosage form is a tablet, the hardness of the tablet may influence the dissolution rate and bioavailability. In order to help achieve the desired dissolution rate and bioavailability, the hardness of the tablet is preferably greater than about 10 Strong Cobb Units (“SCU”). More preferably, the hardness of the tablet is about 15 Strong Cobb Units (“SCU”) to about 30 SCU, and more preferably about 20 SCU to about 28 SCU for a 1000 to 1900 mg tablet. Hardness is typically measured by determining lateral breaking strength (expressed in kiloponds (“kp”) or SCU wherein 1 kp=1.4 S.C.U.)

The compression force applied during tabletting may also influence the dissolution rate and bioavailability. In order to maintain the physical and chemical properties of the API in the final tablet dosage form, the compression force applied to the granulate is preferably selected so as to enable both good physical properties of the tablet (e.g., friability, hardness) and rapid dissolution upon contact of tablet with a hydrophilic medium.

The friability of conventional tablets is measured by the percentage weight loss following a typical friability test. Friability is a standard test known to one skilled in the art. Friability is measured under standardized conditions by weighing out a certain number of tablets (generally 20 or more), placing them in a rotating Plexiglas drum in which they are lifted during replicate revolutions by a radial lever, and then dropped through the diameter of the drum. After replicate revolutions, the tablets are reweighed and the percentage of powder “rubbed off” or broken pieces is calculated. Friability in the range of about 0% to 3% is considered acceptable for most drug and food tablet contexts. Preferably, the friability of the tablet is about 0% to about 1%, more preferably about 0% to about 0.6%, and most preferably less than about 0.3%.

In one preferred embodiment, the invention encompasses a pharmaceutical composition comprising efavirenz, wherein at least about 15% of the total amount of the efavirenz in the composition is dissolved within 15 minutes in a hydrophilic medium (under the USP conditions set forth above), such as about 15% to about 40%. Preferably at least about 40% of the total amount of the efavirenz in the composition is dissolved within 30 minutes, such as about 40% to about 50%, more preferably about 50% or more, such as about 50% to about 70% or about 55% to about 65%. Preferably, the pharmaceutical composition is in the form of a tablet. The above dissolution rates are determined using a composition of efavirenz in a hydrophilic medium having a concentration of about 3.17 ml of medium per each mg of efavirenz under the USP conditions set forth above.

In another embodiment, the invention encompasses a method for treating or preventing a medical condition by administering any of the above-described pharmaceutical compositions to a patient in need thereof.

Having described the invention with reference to certain preferred embodiments, other embodiments will become apparent to one skilled in the art from consideration of the specification. The invention is further defined by reference to the following examples describing in detail the analysis of the crystals and processes for making the crystals of the invention. It will be apparent to those skilled in the art that many modifications, both to materials and methods, may be practiced without departing from the scope of the invention.

### EXAMPLES

The compositions were tested in vitro with dissolution media simulating fasted conditions. The dissolution testing was conducted in a U.S.P. Type II (paddle) apparatus using 935 ml of buffer phosphate pH 6.0 with 0.15% SLS at 37° C. and 50 rpm for 300 mg dose; 1870 ml buffer was used for 600 mg dose. The samples were analyzed using UV at 250 nm for SUSTIVA or HPLC method for the ATRIPLA composition.

It was found that compositions prepared in accordance with the invention exhibited dissolution rates in conditions simulating fasted conditions significantly greater than those compositions prepared by a conventional wet granulation technique. It is expected that the dissolution trends observed in vitro would reflect the trends when tested in vivo.

In the following examples, “EF A” means the polymorphic Form N of efavirenz described in WO 2006/040643, which exhibits a typical X-Ray Powder diffraction having characteristic 2θ values at 7.84, 13.12, 15.04, 18.40, 19.54, 20.82, 25.30 and 25.96, and which is incorporated herein by reference. “EF B” refers to crystalline Form 1 of Efavirenz that is described in U.S. Pat. No. 6,673,372, which is characterized by an x-ray powder diffraction pattern comprising four or more 2θ values selected from the group consisting of 6.0±0.2, 6.3±0.2, 10.3±0.2, 10.8±0.2, 14.1±0.2, 16.8±0.2, 20.0±0.2, 20.5±0.2, 21.1±0.2, and 24.8±0.2, and which is incorporated herein by reference.

### Examples 1 and 2 (Efavirenz Tabs)

### Conventional Wet Granulation

TABLE I

| | | Amount mg/ | |
|---|---|---|---|
| | Amount mg/dose | dose | SUSTIVA |
| Excipient/API | Example 1 | Example 2 | (600 mg) |
| PART I | | | |
| Avicel PH102 | 400.0 | 400.0 | |
| EF A | 600.0 | | |
| EF B | | 600.0 | |
| Sodium Starch | 60.0 | 60.0 | |
| Glycolate | | | |
| Klucel ® LF | 15.0 | 15.0 | |
| Sodium lauryl sulphate | 50.0 | 50.0 | |
| Granulation solution I | | | |
| water | 600.0 | 800.0 | |
| Poloxamer F127 | 10.0 | 10.0 | |
| Granulation solution II | | | |
| water | 750.0 | 750.0 | |
| PART II | | | |
| Avicel PH102 | 335.0 | 335.0 | |
| Sodium Starch | 20.0 | 20.0 | |
| Glycolate | | | |
| PART III | | | |
| Magnesium stearate | 10.0 | 10.0 | |
| Total weight | 1500.0 | 1500.0 | |
| TOP COAT | | | |
| Opadry II | 50.0 | 50.0 | |
| DISSOLUTION | | | |
| 15 min | 23.1% | 29.4% | 42% |
| 30 min | 33.1% | 41.4% | 64% |
| 60 min | 39.5% | 46.4% | 74% |
| 90 min | 44.7% | 51.4% | 80% |
| 180 min | 52.5% | 60.3% | 86% |

Part I ingredients were mixed by a high shear mixer then wet granulated with granulation solution I (poloxamer dissolved with water) followed by granulation solution II, using high shear mixer. The resulting granules were then dried in a fluidized bed drier. The granulate was milled in Frewitt 0.6. Part II ingredients were then added to the milled granulate and mixed in Y-cone for 10 minutes to form a dry blend. The magnesium stearate of Part III was milled with 50 mesh then added to the dry blend and mixed in Y-cone for 5 minutes to form a final composition, which was then compressed into tablets at 28 SCU.

The dissolution profiles of both batches of Example 1 and 2 were tested in dissolution media simulating the GI conditions in the fasted state, and compared to the dissolution profile of SUSTIVA® 600 mg efavirenz tablets (Bristol-Myers Squibb). The results are illustrated in Table I, above.

### Examples 3 and 4

### Conventional Dry Granulation and Direct Compression

TABLE II

| | | Amount mg/ |
|---|---|---|
| | Amount mg/dose | dose |
| Excipient/API | Example #3 | Example # 4 |
| PART I | | |
| Lactose anhydrous | 400.0 | 400.0 |
| EF A | 600.0 | |
| EF B | | 600.0 |
| Aerosil | 20.0 | 20.0 |
| Sodium starch glycolate | 60.0 | 60.0 |
| PART II | | |
| Sodium lauryl sulphate | 50.0 | 50.0 |
| Starch NF | 30.0 | 30.0 |
| PART III | | |
| Magnesium stearate | 5.0 | 5.0 |
| PART IV | | |
| Avicel PH102 | 310.0 | 310.0 |
| Sodium Starch | 20.0 | 20.0 |
| Glycolate | | |
| PART V | | |
| Magnesium stearate | 5.0 | 5.0 |
| Total weight | 1500.0 | 1500.0 |
| TOP COAT | | |
| Opadry II | 50.0 | 50.0 |
| DISSOLUTION | | |
| 15 min | 21.9% | 28.0% |
| 30 min | 33.0% | 36.1% |
| 60 min | 39.7% | 48.1% |
| 90 min | 44.8% | 56.0% |
| 180 min | 52.7% | 66.0% |

A mixture of Part I ingredients were sieved through 30 mesh sieve, followed by addition of part II ingredients. The mixture was mixed in Y-cone for 10 minutes. Subsequently, magnesium stearate of part III was sieved through 50 mesh and added to the mixture and mixed for additional 5 minutes in Y-cone. The mixture was compressed into slugs of 1050-1070 mg weight in the RTS with 20 mm stamps to give hardness of 16-18 SCU. The slugs were sieved using Frewitt 0.6 mm. The granulate was mixed with part IV ingredients in the Y-cone for 10 minutes followed by addition of 50 mesh sieved magnesium stearate of part V and mixed for 5 minutes. Tablet with 25 SCU were made from the final blend. The dissolution profiles of both batches of Example 3 and 4 were tested in dissolution media simulating the GI conditions in the fasted state, and compared to the dissolution profile of SUSTIVA® 600 mg efavirenz tablets (Bristol-Myers Squibb). The results are illustrated in Table II.

### Examples 5-9

### Wet Milling/Spray Granulation

TABLE III

| | Amount mg/ | Amount | Amount | Amount |
|---|---|---|---|---|
| | dose | mg/dose | mg/dose | mg/dose |
| Excipient/API | Example 5 | Example 6 | Example 7 | Example 8 |
| PART I EF | | | | |
| dispersion | | | | |
| Klucel ® LF | 50.0 | 70.0 | 80.0 | 110.0 |
| Purified water | 2815.0 | 2815.0 | 2815.0 | 2815.0 |
| EF A | 600.0 | 600.0 | 600.0 | 600.0 |
| EF B | | | | |
| PART II spray | | | | |
| granulation | | | | |
| Avicel PH 101 | 200 | 80.0 | 200.0 | 200.0 |
| Lactose monohydrate | 200 | 80.0 | 200.0 | 200.0 |
| NF/BP (100 mesh) | | | | |
| Starch 1500 NF | 64.0 | 25.0 | 64.0 | 64.0 |
| (pregelatinised) | | | | |
| Primogel | 120.0 | 48.0 | 120.0 | 120.0 |
| EF (as sprayed | 600.0 | 600.0 | 600.0 | 600.0 |
| dispersion) | | | | |
| Klucel ® (as sprayed | 50.0 | 70.0 | 80.0 | 110.0 |
| dispersion) | | | | |
| PART III dry mix | | | | |
| Granulate from step | 1270.0 | 1191.0 | 1184.0 | 1532.0 |
| II | | | | |
| Lactose | 50.0 | 100.0 | 100.0 | |
| monohydrate NF/BP | | | | |
| (100 mesh) | | | | |
| Avicel PH 101 | 35.0 | 70.0 | 70.0 | |
| Sodium bicarbonate | 75.0 | 75.0 | 75.0 | 48.0 |
| Tartaric Acid | 50.0 | 50.0 | 50.0 | 32.0 |
| Aerosil 200 | | | | 10.0 |
| Part IV Dry mix | | | | |
| Aerosil 200 | 10.0 | 10.0 | 10.0 | |
| Part V Dry mix | | | | |
| Magnessium | 10.0 | 10.0 | 10.0 | 10.0 |
| stearate | | | | |
| Total weight | 1500.0 | 1506.0 | 1499.0 | |
| DISSOLUTION | | | | |
| 15 min | 65.2% | 62.9% | 61.9% | 46.4% |
| 30 min | 79.4% | 76.9% | 75.5% | 53.8% |
| 60 min | 80.8% | 79.9% | 76.8% | 55.7% |
| 90 min | 83.2% | 80.8% | 79.9% | 57.4% |
| 180 min | 82.7% | 80.8% | 81.1% | 59.5% |

KLUCEL® (of part I) was dissolved in purified water (of part I) to form Klucel® solution. The EF (of part I) was dispersed in the Klucel® solution using mixer, followed by homogenizer for about 15 minutes, to form Efavirenz dispersion I. Dispersion I was then homogenized using high pressure homogenization process (MFIC microfluidizer M-110F) to form efavirenz dispersion II.

Efavirenz dispersion II was then sprayed granulated on part II ingredients using a fluid-bed-top-spray-granulation process.

The final granulate was tested for assay correction.

The final granulate was mixed with the ingredients of step 3, followed by mixing with part IV ingredient. Finally magnesium stearate of part V was mixed with the granulate to form a final composition and the composition was compressed into tablets.

The dissolution profiles of both batches of Example 5 and 8 was tested in dissolution media simulating the GI conditions in the fasted state, and compared to the dissolution profile of SUSTIVA® 600 mg efavirenz tablets (Bristol-Myers Squibb). The results are illustrated in Table III.

### Examples 10 and 11

### Conventional Wet Granulation Composition of ATRIPLA

TABLE IV

| | | Amount mg/ |
|---|---|---|
| | Amount mg/dose | dose |
| Excipient/API | Example 10 | Example 11 |
| PART I | | |
| EF A | 600.0 | |
| EF B | | 600.0 |
| Aerosol 200 | 10.0 | 10.0 |
| Avicel PH 102 | 240.0 | 240.0 |
| PART II | | |
| Sodium starch glycolate | 60.0 | 60.0 |
| klucel | 15.0 | 15.0 |
| Granulation solution I | | |
| Lutrol F127 (poloxamer | 20.0 | 20.0 |
| NF, 407) | | |
| water | 355 | 356 |
| PART III | | |
| Mannitol USP/BP | 50.0 | 50.0 |
| Emtricitabine | 200.0 | 200.0 |
| Lactose 200 mesh | 90.0 | 90.0 |
| Crospovidone | 20.0 | 20.0 |
| Granulation solution II | | |
| PVP k-30 | 10.0 | 10.0 |
| water | 37.5 | 62.5 |
| PART IV | | |
| Magnesium stearate | 10.0 | 10.0 |
| PART V | | |
| Lactose spray dried | 100.0 | 100.0 |
| Tenofovir Disoproxil | 300.0 | 300.0 |
| Fumarate | | |
| Crospovidone | 40.0 | 40.0 |
| PART VI | | |
| Magnesium stearate | 10.0 | 10.0 |
| Total weight | 1775.0 | 1775.0 |
| TOP COAT | | |
| Opadry II 85F34156 | 53.25 | 53.25 |
| pink | | |
| DISSOLUTION | | |
| 15 min | 27.8% | 41.5% |
| 30 min | 35.3% | 57.9% |
| 60 min | 41.6% | 67.5% |
| 90 min | 51.4% | 76.5% |
| 180 min | 53.7% | 77.0% |

Part I ingredients were sieved through 30 mesh (Example 11 only) followed by mixing in a high shear mixer. Subsequently, Part II ingredients were added and mixed together in a high shear mixer. Subsequently, granulation solution I was added (poloxamer dissolved with water) followed by quadro co-mill sieving, i.e., sieving during co-milling. The granulate was dried with fluid bed drier and sieved with Frewit 0.63 mm.

Part III ingredients were mixed in a high shear mixer followed by wet granulation with granulation solution II, (PVP in water) using high shear mixer, followed by co-mill sieving. The resulting granules were then dried in a fluidized bed drier and milled in Frewitt 0.6. Part III milled granulate was mixed in Y-cone for 15 min.

Part IV ingredient magnesium stearate (presieved with 50 mesh) was then added to the mixed granulate and mixed in Y-cone for additional 5 minutes to form a dry blend.

Part V ingredients were milled with 30 mesh and then mixed in Y-cone for 20 minutes. Part IV was then added to the dry blend and mixed in Y-cone for 20 minutes. Part VI ingredient magnesium stearate (presieved with 50 mesh) was then added to the mixed granulate and mixed in Y-cone for an additional 5 minutes to form a dry blend.

The resulting blend was then mixed with efavirenz granulate from part II in Y-cone for 15 minutes to form a final composition, which was then compressed into tablets at 33 SCU for example 11 and 19 SCU for example 10.

The dissolution profiles of both batches of Example 10 and 11 were tested in dissolution media simulating the GI conditions in the fasted state, and compared to the dissolution profile of ATRIPLA® 600 mg efavirenz, 200 mg Emtricitabine and 300 mg tenofovior tablets (Bristol-Myers Squibb). The results are illustrated in Table IV, above.

### Example 12

### Wet Milling/Spray Granulation Composition of ATRIPLA®

TABLE V

| | Amount mg/ | |
|---|---|---|
| | dose | Amount mg/dose |
| Excipient/API | Example 12 | Atripla ® |
| PART I EF dispersion | | |
| Klucel ® LF | 70.0 | |
| Purified water | 2815.0 | |
| EF A | 600.0 | |
| EF B | | |
| PART II spray | | |
| granulation | | |
| Avicel PH 101 | 80.0 | |
| Lactose monohydrate | 80.0 | |
| NF/BP (100 mesh) | | |
| Starch 1500 NF | 25.0 | |
| (pregelatinised) | | |
| Primogel | 48.0 | |
| EF (as sprayed | 600.0 | |
| dispersion) | | |
| Klucel (as sprayed | 70.0 | |
| dispersion) | | |
| PART III dry mix | | |
| Granulate from step II | 950.0 | |
| Aerosil 200 | 10.0 | |
| Sodium bicarbonate | 48.0 | |
| Tartaric Acid | 32.0 | |
| Part IV Dry mix | | |
| Magnessium stearate | 10.0 | |
| Total weight | 1050.0 | |
| Part V Dry mix | | |
| Emtricitabine | 200.0 | |
| Tenofovir | 300.0 | |
| Avicel PH 101 | 89.5 | |
| Primogel | 48.0 | |
| Part VI Dry mix | | |
| Magnessium stearate | 10.0 | |
| Total weight | 647.5 | |
| Part VII dry mix | | |
| Mix from part IV | 1050.0 | |
| Mix from part VI | 647.5 | |
| Total weight | 1697.5 | |
| DISSOLUTION | | |
| 15 min | 59.2% | 51.0% |
| 30 min | 80.4% | 71.8% |
| 60 min | 84.4% | 79.0% |
| 90 min | 85.4% | 85.1% |
| 180 min | 89.1% | 92.1% |

KLUCEL® (of part I) was dissolved in purified water (of part I) to form Klucel® solution. The EF (of part I) was dispersed in the Klucel® solution using mixer, followed by homogenizer for about 15 minutes, to form Efavirenz dispersion I. Dispersion I was then homogenized using high pressure homogenization process (MFIC microfluidizer M-110F) to form efavirenz dispersion II.

Efavirenz dispersion II was then a sprayed granulated on part II ingredients using a fluid-bed-top-spray-granulation process.

The final granulate was tested for assay correction.

The final granulate from part 2 were mixed with the ingredients of part 3. Finally magnesium stearate of part IV was mixed with the granulate to form a final composition.

Part V ingredient was mixed together, followed by mixing with magnesium stearate (part VI).

The two blends from part IV and part VI were mixed and the composition was compressed into tablets.

The dissolution profiles of Example 12 was tested in dissolution media simulating the GI conditions in the fasted state, and compared to the dissolution profile of ATRIPLA® 600/300/200 mg efavirenz, emtricitabine tenofovir tablets (Bristol-Myers Squibb). The results are illustrated in Table V.

### Summary of Examples

### Efavirenz Tabs

| | | | API | % dissolved |
|---|---|---|---|---|
| Example | Sample description | Process | type | at 30 min |
| 1 | Efavirenz tabs - | Wet | A | 33.1% |
| | conventional process | granulation | | |
| 2 | Efavirenz tabs - | Wet | B | 41.4% |
| | conventional process | granulation | | |
| 3 | Efavirenz tabs - | Direct | A | 33.3% |
| | conventional process | compression | | |
| 4 | Efavirenz tabs - | Direct | B | 36.1% |
| | conventional process | compression | | |
| 5 | Efavirenz tabs - process | Process of | A | 79.4% |
| | of invention | invention | | |
| 6 | Efavirenz tabs - process | Process of | A | 76.9% |
| | of invention | invention | | |
| 7 | Efavirenz tabs - process | Process of | A | 75.5% |
| | of invention | invention | | |
| 8 | Efavirenz tabs - process | Process of | A | 53.8% |
| | of invention | invention | | |

### Summary of Examples

### Efavirenz/Emtricitabine/Tenofovir Tabs

| Example | | | API | % dissolved |
|---|---|---|---|---|
| no | Sample description | Process | type | at 30 min |
| 10 | Efavirenz tabs - | Wet | A | 35% |
| | conventional process | granulation | | |
| 11 | Efavirenz tabs - | Wet | B | 57% |
| | conventional process | granulation | | |
| 12 | Efavirenz tabs - process | Process of | A | 80% |
| | of invention | invention | | |

What is claimed is:

1. A method for preparing a pharmaceutical composition, said pharmaceutical composition comprising at least 30% of efavirenz by weight of the total composition excluding the weight of an optional coating, comprising co-milling efavirenz with a hydrophilic polymer.

2. The method of claim 1, wherein said pharmaceutical composition comprises about 300 mg or more of efavirenz.

3. The method of claim 2, wherein said pharmaceutical composition comprises about 300 to about 800 mg of efavirenz.

4. The method of claim 3, wherein said pharmaceutical composition comprises about 300 to about 600 mg of efavirenz.

5. The method of any of claims 1 to 3, wherein said pharmaceutical composition does not comprise a surfactant and wherein the co-milling step is wet co-milling.

6. The method of claim 5, further comprising spray granulating the obtained co-milled composition.

7. The method of any of claims 1 to 6, wherein the hydrophilic polymer is selected from the group consisting of methacrylic acid co-polymer, polyvinylpyrrolidone, polyethylene glycol, polyvinyl alcohol, vinylpyrrolidone/vinylacetate, hypromellose, hydroxypropylcellulose, carboxymethylethylcellulose, hydroxypropylmethylcellulose phthalate, hydrolyzed collagens, and mixtures thereof.

8. The method of claim 7, wherein said hydrophilic polymer is present in an amount of about 0.2% to about 20% by weight of the total composition excluding the weight of an optional coating.

9. The method of claim 7, wherein the hydrophilic polymer is hydroxypropylcellulose.

10. The method of claim 9, wherein said hydroxypropylcellulose is present in an amount of about 3% to about 4% by weight of the total composition excluding the weight of an optional coating.

11. The method of any of claims 1 to 10, wherein the hydrophilic polymer is present in an amount of about 0.5% to about 20% by weight of the total composition.

12. The method of claim 11, wherein the hydrophilic polymer is present in an amount of about 2% by weight of the total composition.

13. The method of any of claims 1 to 12, further comprising mixing the co-milled composition with a carbonate or bicarbonate and at least one pharmaceutically acceptable acid.

14. The method of claim 13, wherein said carbonate is sodium or potassium carbonate.

15. The method of claim 13, wherein said bicarbonate is selected from the group consisting of ammonium bicarbonate, an alkali metal bicarbonate or an alkaline earth metal bicarbonate.

16. The method of claim 15, wherein said bicarbonate is sodium, magnesium, or potassium bicarbonate.

17. The method of any of claim 13 to 16, wherein said pharmaceutically acceptable acid is selected from the group consisting of ascorbic acid, citric acid, tartaric acid, succinic acid, fumaric acid, malic acid, lactic acid, propionic acid, sorbic acid, and benzoic acid.

18. The method of claim 17, wherein said pharmaceutically acceptable acid is tartaric acid.

19. The method of any of the preceding claims, wherein at least 50% of the total amount of said efavirenz in said composition dissolves within 30 minutes in a U.S.P. Type II (paddle) apparatus using buffer phosphate pH 6.0 with 0.15% SLS at 37° C. at 50 rpm and in an amount of 3.17 ml buffer per mg of efavirenz.

20. A pharmaceutical composition prepared by the method of any of claims 1 to 19.

21. A pharmaceutical composition, comprising a co-milled composition of efavirenz with at least one hydrophilic polymer, wherein said efavirenz is present in an amount of at least 30% by weight of the pharmaceutical composition, excluding the weight of an optional coating, wherein said pharmaceutical composition does not comprise a surfactant.

22. The pharmaceutical composition of claim 16, wherein said pharmaceutical composition comprises about 300 mg or more of efavirenz.

23. The pharmaceutical composition of claim 22, wherein said pharmaceutical composition comprises about 300 to about 800 mg of efavirenz.

24. The pharmaceutical composition of claim 22, wherein said pharmaceutical composition comprises about 300 to about 600 mg of efavirenz.

25. The pharmaceutical composition of claims 21 to 24, wherein about 40% to about 50% of the total amount of efavirenz in said composition dissolves within about 30 minutes in a U.S.P. Type II (paddle) apparatus using buffer phosphate pH 6.0 with 0.15% SLS at 37° C. at 50 rpm and in an amount of 3.17 ml buffer per mg of efavirenz.

26. The pharmaceutical composition of claim 25, wherein about 50% to about 70% of the total amount of efavirenz in said composition dissolves within about 30 minutes.

27. The pharmaceutical composition of claim 26, wherein about 55% to about 65% of the total amount of efavirenz in said composition dissolves within about 30 minutes.

28. The pharmaceutical composition of any of claims 21 to 27, further comprising at least one carbonate or bicarbonate, and at least one pharmaceutically acceptable acid.

29. The pharmaceutical composition of any of claims 21 to 27, further comprising an alkali metal carbonate and at least one pharmaceutically acceptable acid.

30. The pharmaceutical composition of claim 29, wherein said alkali metal carbonate is sodium or potassium carbonate.

31. The pharmaceutical composition of claim 28, wherein said bicarbonate is selected from the group consisting of ammonium bicarbonate, an alkali metal bicarbonate and an alkaline earth metal bicarbonate.

32. The pharmaceutical composition of claim 31, wherein said bicarbonate is sodium, magnesium, or potassium bicarbonate.

33. The pharmaceutical composition of any of claims 28 to 32, wherein said pharmaceutically acceptable acid is selected from the group consisting of ascorbic acid, citric acid, tartaric acid, succinic acid, fumaric acid, malic acid, lactic acid, propionic acid, sorbic acid, and benzoic acid.

34. The pharmaceutical composition of claim 33, wherein said pharmaceutically acceptable acid is tartaric acid.

35. The pharmaceutical composition of and of claims 21 to 34, wherein the hydrophilic polymer is selected from the group consisting of methacrylic acid co-polymer, polyvinylpyrrolidone, polyethylene glycol, polyvinyl alcohol, vinylpyrrolidone/vinylacetate, hypromellose, hydroxypropylcellulose, carboxymethylethylcellulose, hydroxypropylmethylcellulose phthalate, hydrolyzed collagens, and mixtures thereof.

36. The pharmaceutical composition of claim 35, wherein the hydrophilic polymer is present in an amount of about 0.2% to about 20% by weight of the total composition.

37. The pharmaceutical composition of claim 35, wherein the hydrophilic polymer is hydroxypropylcellulose.

38. The pharmaceutical composition of claim 37, wherein said
hydroxypropylcellulose is present in an amount of about 3% to about 4% by weight of the total composition.

39. A method for treating or preventing a medical condition in a patient, comprising administering to said patient the pharmaceutical composition of any one of claims 21 to 39.

40. A pharmaceutical composition according to claim 39 wherein the medical condition is an HIV-1 infection.
